(19) United States
(12) Patent Application Publication
(10) Pub. No.: US 2017/0028100 A1
(43) Pub. Date: Feb. 2, 2017

(54) BIOLOGICAL FIBER MEMBRANE AND METHOD FOR PREPARING THE SAME

(71) Applicant: HARVEST BELLE BIOTECH, New Taipei City (TW)

(72) Inventor: Chi-Hsiang Lu, New Taipei City (TW)

(21) Appl. No.: 14/812,257

(22) Filed: Jul. 29, 2015

(51) Int. Cl. A61L 26/00; A61F 13/00; C12P 1/04

(52) U.S. Cl. CPC A61L 26/0057; C12P 1/04; A61L 26/0095; A61F 13/00063; A61L 2300/608; A61F 13/122

(57) ABSTRACT

The present invention provides a biological fiber membrane formed by bacteria of the genus Gluconacetobacter. The biological fiber membrane includes a first surface layer and an opposing second surface layer and a three-dimensional reticular structure bound between the first surface layer and the second surface layer, such that the moisturizing property of the biological fiber membrane is improved, thereby carrying more active ingredients. The present invention further provides a method for preparing a biological fiber membrane.

### BACKGROUND OF THE INVENTION

1. Field of the Invention

The present invention relates to biological fiber membranes, and more particularly, to a biological fiber membrane applied to the skin.

2. Description of the Related Art

In the current medical field, a cotton pad or gauze is mostly used as the dressing for the care of a wound. However, such type of dressing has some drawbacks, for example, a poor antibacterial property, a high possibility of a wound infection, easiness for developing wound adhesion, and difficulty to be removed.

Afterwards, the cotton pads and gauzes have been replaced with non-woven dressings, since the non-woven dressings have the characteristics of better absorbency and being capable of providing a moist environment to aid of the wound repair. However, when the liquid or moisture absorbed by the non-woven dressings is gradually reduced, the issue of wound adhesion is likely to arise.

On the other hand, in addition to the basic living requirements, the modern people pay more attention to cosmetic skin care, especially for the facial care. Hence, the beauty industry focuses on the demands of the facial care, and develops a variety of facial mask products. There are a variety of masks, such as a mud paste-type mask, a tear-peel type mask, a sheet-like mask, and so on.

Although the mud paste-type mask contains some ingredients or minerals for skin care, the mask has to be washed off after application. Hence, the ingredients for skin care are hard to be really absorbed by the skin. Further, because the mud paste-type mask contains more minerals, more preservatives must be added to prevent bacteria from growing in the moist mud paste. The tear-peel type mask has main ingredients, such as, polymer gel, water and alcohol, and promotes the blood circulation of the skin by increasing the epidermal temperature. However, since the tear-peel type mask is no peeled off until being dry, it might cause damage to the sensitive skin during the peeling of the mask. In addition, the tear-peel-type mask does not contain any moisturizing ingredients for the dryness of the mask, such that it is not appropriate for the dry skin. The sheet-like mask is a monolayer sheet absorbed with essence with specific functions, and it can be used for a variety of skin cares by adjusting the ingredients. Although the sheet-like mask does not need to be washed off after application, the mask does not have any cleaning effect. The above sheet-like mask is mostly made of a monolayer of non-woven fabric. For a user to apply the non-woven fabric soaked with the essence, a higher concentration of essence is required due to the rapid water evaporation in the non-woven fabric. Consequently, the essence is wasted, while the problem of water evaporation remains unresolved.

Therefore, there is still a need to develop a novel dressing product.

### SUMMARY OF THE INVENTION

In view of the above disadvantages of the prior art, the present invention provides a biological fiber membrane formed by bacteria of the genus Gluconacetobacter, and the biological fiber membrane includes a first surface layer and a second surface layer, and a three-dimensional reticular structure bound between the first surface layer and the second surface layer, wherein the density of the three-dimensional reticular structure is smaller than the density of the first surface layer and the second surface layer.

In one embodiment, the three-dimensional reticular structure is composed of a plurality of biological fibers.

Further, the three-dimensional reticular structure has a plurality of backbone fibers parallel to each other and a plurality of inter-layer fibers interwoven at any two of the adjacent backbone fibers. Both of the backbone fiber and the inter-layer fiber are biological fibers, and the diameter of each of the backbone fibers is greater than or equal to the diameter of each of the inter-layer fibers.

In yet one embodiment, the biological fiber membrane further includes an active ingredient or a drug. The active ingredient can be a humectant, a whitening ingredient, an anti-wrinkle ingredient, an exfoliating ingredient, a growth factor or an enzyme.

The present invention further provides a method for preparing a biological fiber membrane, and the method includes steps of providing a container having a culture medium, wherein the culture medium has a carbon source, a peptone and a yeast extract at a weight ratio from 5:1:1 to 4:1:1; and culturing bacteria of the genus Gluconacetobacter in the culture medium for 24 hours to 96 hours.

The biological fiber membrane of the present invention is formed by bacteria of the genus Gluconacetobacter. There is a three-dimensional reticular structure bound between the first surface layer and the second surface layer of the biological fiber membrane, such that the moisturizing property of the biological fiber membrane is improved, and thereby being capable of carrying more active ingredients.

### BRIEF DESCRIPTION OF THE DRAWINGS

FIG. 1 is a schematic diagram of a biological fiber membrane of the present invention;

FIG. 2A shows a scanning electron microscope (SEM) photograph of the three-dimensional reticular structure of the biological fiber membrane of the present invention;

FIG. 2B shows a side view of the biological fiber membrane of the present invention;

FIGS. 3A and 3B show an SEM photograph at 500× magnification of the biological fiber membrane of the present invention and an SEM photograph at 500× magnification of the conventional biological fiber membrane, respectively; and

FIG. 4 shows results of a permeability test on the biological fiber membrane of the present invention and the conventional biological fiber membrane, wherein FIG. 4(a) shows the test result of the biological fiber membrane of the present invention, and FIG. 4(b) shows the test result of the conventional biological fiber membrane.

### DETAILED DESCRIPTION OF THE PREFERRED EMBODIMENTS

The following specific examples are used for illustrating the present invention. One skilled in the art can easily conceive the other advantages and effects of the present invention, from the disclosure of the present specification.

It should be noted that all of the drawings depict a structure, proportion, size, etc., are only used to match the specification for a person skilled in the art to understand and reads. It is not intended to limit the conditions which can be implemented in the present invention, such that it is not substantially meaningful technically. Any modification of the structure, change in the proportion or adjustment of the size are be within the scope encompassed in the technical contents disclosed in the present invention, without departing from the spirit of the present invention. At the same time, terms, such as “first,” “second,” “on” and “a/an” etc., are merely to facilitate the understanding of the descriptions, and should not be construed to limit the implemental scope of the present invention. Any change or adjustment of the relationships is also considered to be within the implemental scope, without any substantial changes to the technical content.

The term “parallel” used herein means the morphology of which a plurality of backbone fibers are in the same direction, such as a longitudinal direction or a width direction.

The present invention provides a biological fiber membrane formed by bacteria of the genus Gluconacetobacter, and the biological fiber membrane includes a first surface layer, an opposing second surface layer, and a three-dimensional reticular structure bound between the first surface layer and the second surface layer. The density of the three-dimensional reticular structure is smaller than the density of the first surface layer and the second surface layer.

The biological fiber membrane of the present invention is obtained by culturing microorganisms. It is found in the present invention found that the biological fiber membrane formed by culturing bacteria of the genus Gluconacetobacter in a culture medium containing mannitol, peptone, yeast extract and agar has a plurality of biological fibers being wound and combined into a three-dimensional structure.

In an embodiment for producing the biological fiber membrane, the fermentation of the bacterial strain takes place. First of all, a culture medium is provided in a container, and the culture medium contains some known components selected from gelatin, gum arabic, agar, and etc. The culture medium still needs some carbon sources, such as mannitol, glucose, and a combination thereof, and other components, such as peptone and yeast extract. The weight ratio of the carbon source, peptone and yeast extract is in a range from 5:1:1 to 4:1:1. Subsequently, the pH value of the culture medium is preferably controlled at acidic, such as between pH 0.5 to 6. The initial concentration of microorganisms can be controlled in a range of 102 to 105 bacteria/ml. The stationary culturing of the microorganisms is performed at 25 to 28° C. for 24 hours to 96 hours. Since a flat container can be used, the three-dimensional reticular structure is flat. After 24 hours to 72 hours, a membrane is taken out, and the biological fiber membrane of the present invention is obtained.

After testing, the thickness of the biological fiber membrane is at least 20 μm, such as from 20 μm to 30 μm, or for example, from 20 μm to 26 μm, or from 24 μm to 26 μm. The diameter of the biological fiber of the biological fiber membrane of the present invention is from about 20 nm to 100 nm. Further, the amount of the biological fibers per unit area is from 0.005 to 0.008 g/cm².

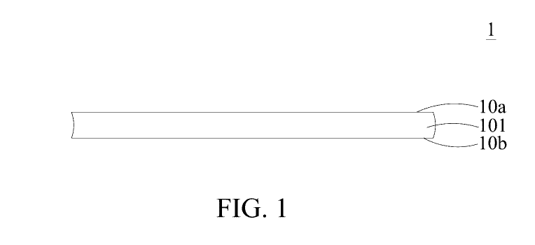

As shown in FIG. 1, a biological fiber membrane 1 of the present invention has a first surface layer 10a and an opposing second surface layer 10b; and a three-dimensional reticular structure 101 bound between the first surface layer 10a and the second surface layer 10b.

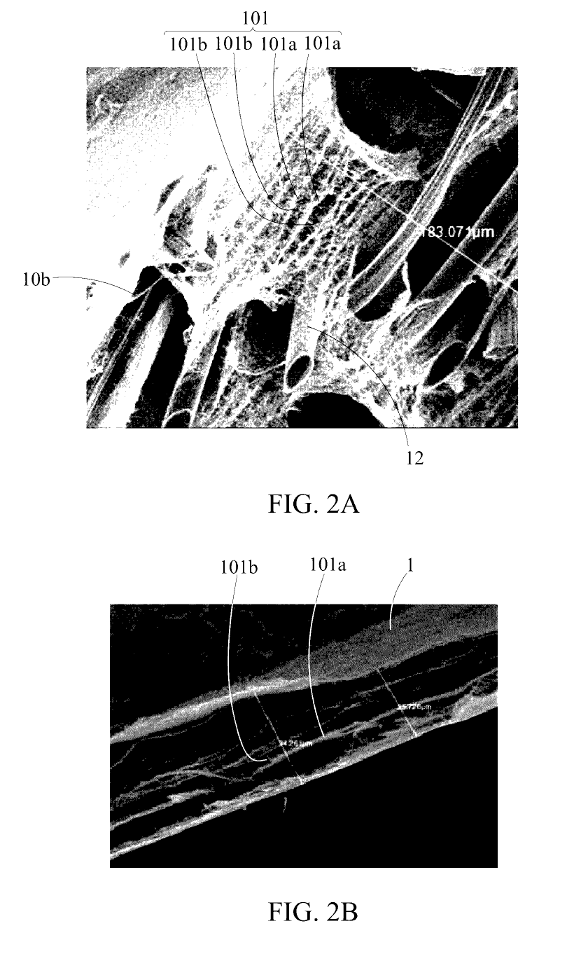

In addition, as shown in FIG. 2A, the three-dimensional reticular structure 101 extends along the second surface layer 10b, and is combined on a cloth membrane fiber 12.

As shown in FIG. 2A, the three-dimensional reticular structure 101 extends to the cloth membrane fiber 12 from the second surface layer 10b, and the three-dimensional reticular structure 101 is composed of a plurality of biological fibers. More particularly, the three-dimensional reticular structure 101 has a plurality of backbone fibers 101a parallel to each other and a plurality of inter-layer fibers 101b interwoven at any two of the adjacent backbone fibers 101a. Therefore, any two of the adjacent backbone fibers 101a are linked to form the three-dimensional reticular structure 101 in the horizontal and vertical directions. Both of the backbone fiber 101a and the inter-layer fiber 101b are biological fibers. As shown in FIG. 2A, the diameter of each of the backbone fibers 101a is greater than the diameter of each of the inter-layer fibers 101b.

As shown in FIG. 2B, a side view of the biological fiber membrane 1 is provided, wherein the biological fiber membrane 1 also has a plurality of backbone fibers 101a, which are parallel to each other or extend along a longitudinal direction or a width direction of the biological fiber membrane 1, and a plurality of inter-layer fibers 101b interwoven with the backbone fibers 101a. In this example, the thickness of the biological fiber membrane 1 is from 20 μm to 30 μm. Further, as shown in FIG. 2B, the density of the three-dimensional reticular structure is smaller than the density of the two surface layers of the biological fiber membrane 1. An active ingredient or a drug can be absorbed between the two surface layers, such that a moisturizing effect is provided.

In addition, a substrate can be used for preparing the biological fiber membrane and enable the membrane to be combined temporarily or permanently. Such method can omit the step of flipping the flat three-dimensional reticular structure over in the container. For example, in the example of FIG. 2A, a cloth membrane is used as a substrate.

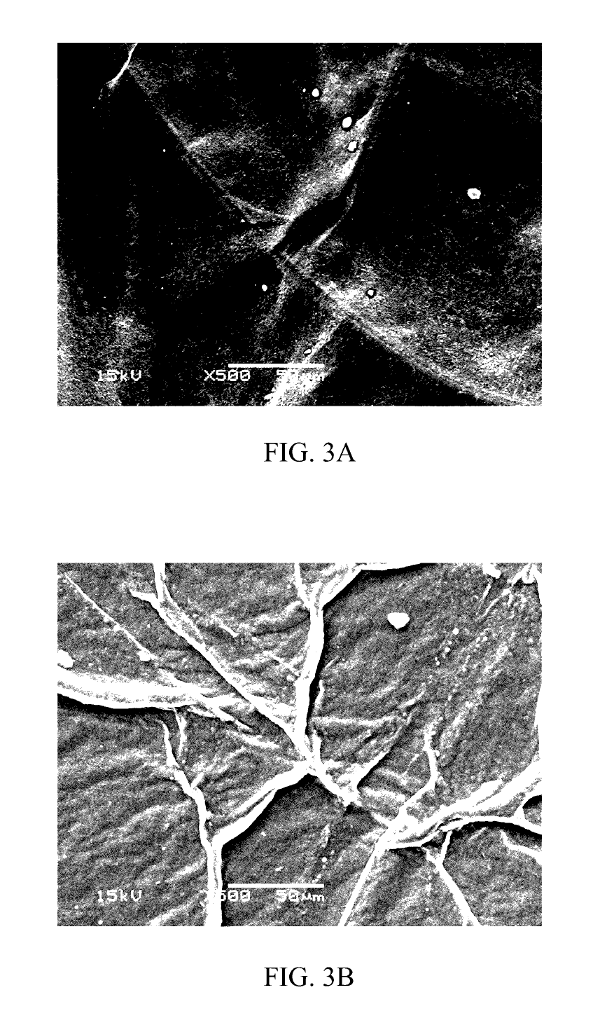

As shown in FIGS. 3A and 3B, an SEM photograph at 500× magnification of the biological fiber membrane of the present invention and an SEM photograph at 500× magnification of the conventional biological fiber membrane are shown respectively.

As shown in FIG. 3A, the surface of the biological fiber membrane of the present invention is flat. The strips shown in FIG. 3A are fibers of the cloth membrane substrate underneath the biological fiber membrane. Hence, the surface of the biological fiber membrane of the present invention is very flat. On the contrary, the surface of the conventional biological fiber membrane shown in FIG. 3B has many foldings. Therefore, poor attachment of such biological fiber membrane to the skin affects touch, resulting in poor absorbency of a drug or an active ingredient.

### Measurements for the Amount of the Biological Fibers Per Unit Area and Absorbency in the Biological Fiber Membrane

The biological fiber membrane of the present invention was cut into 16 pieces, each with a size of 5 cm×5 cm. These pieces were dried at 60° C. for 10 minutes, and then the dry weight of these pieces were weighed. The result for each of the pieces was obtained by dividing the dry weight by the area of the piece, and the measured amount of the biological fibers per unit area was from 0.005 to 0.008 g/cm².

Further, these pieces were soaked in water for 5 minutes to weigh the wet weights. The water content per unit area was measured by the following equation:

water content per unit area=(wet weight−dry weight)/area

After testing, the water content per unit area of the biological fiber membrane of the present invention was from 0.06 to 0.14 g/cm².

In comparison, the amount of the biological fibers per unit area of the conventional biological fiber membrane was only 0.001 g/cm² and the water content of the conventional biological fiber membrane was only 0.04 g/cm². Therefore, the biological fiber membrane of the present invention is capable of carrying more water or active ingredients, such that the moisturizing effect achieved is better.

### Permeability Test on the Biological Fiber Membrane of the Present Invention

The biological fiber membrane of the present invention and the conventional biological fiber membrane were placed flat on a substrate, and then the same amount stained essences was added onto the biological fiber membrane of the present invention and the conventional biological fiber membrane, respectively. After 10 seconds, the permeability of the substrate was observed visually.

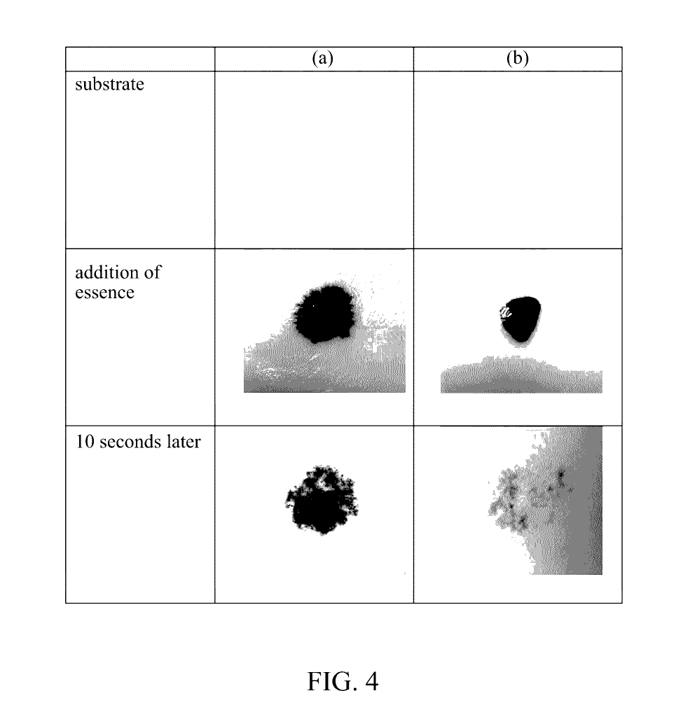

Referring to FIG. 4, FIG. 4(a) shows the test result of the biological fiber membrane of the present invention and FIG. 4(b) shows the test result of the conventional biological fiber membrane. Among these results, when the essences were just added onto the conventional biological fiber membrane, the essences had poor spreading. After 10 seconds, the essences could not penetrate effectively through the substrate. By contrast, the biological fiber membrane of the present invention has better diffusibility and significant permeability.

In summary, the biological fiber membrane of the present invention is provided to apply to the skin. It is particularly useful as a mask. The biological fiber membrane of the present invention further includes an active ingredient or a drug. Before the end of fermentations of a bacterial strain, the active ingredient or drug is added to the three-dimensional reticular structure. Upon completing the preparation of the biological fiber membrane, the active ingredient or drug is included in the membrane. The examples of the active ingredient include humectants, whitening ingredients, anti-wrinkle ingredients, exfoliate ingredients, growth factors and enzymes. The examples of the drug include massage drugs in the form of pastes or liquids, such as massage oil or stress-relieving oil. Because the biological fiber membrane of the present invention has a three-dimensional reticular structure, the active ingredient or drug is not easily exposed. After applying the biological fiber membrane of the present invention to the body or skin and pressing the biological fiber membrane by massage or other contact devices, the release of the active ingredient or drug is facilitated. In addition, the release of the active ingredient or drug is also facilitated by heating.

While the examples are used to illustrate the principle of the present invention and the effect being brought about, they are not intended to limit the preset invention. Any one skilled in the art can make modifications to the examples above without substantially departing from the spirit and scope of the present invention. Therefore, the scope of the present invention should be accorded to the appended claims.

1. A biological fiber membrane formed by culturing bacteria of the genus Gluconacetobacter in a culture medium having mannitol, peptone, yeast extract and agar, comprising:
a first surface layer and an opposing second surface layer; and
a three-dimensional reticular structure bound between the first surface layer and the second surface layer, wherein the density of the three-dimensional reticular structure is smaller than the density of the first surface layer and the second surface layer, wherein the three-dimensional reticular structure has a plurality of backbone fibers parallel to each other and a plurality of inter-layer fibers interwoven at any two of adjacent backbone fibers, and the diameter of each of the plurality of backbone fibers is greater than or equal to the diameter of each of the plurality of inter-layer fibers, and wherein the plurality of backbone fibers extend along a longitudinal direction or a width direction of the biological fiber membrane.

2. The biological fiber membrane of claim 1, wherein the three-dimensional reticular structure is composed of a plurality of biological fibers.

3. (canceled)

4. The biological fiber membrane of claim 1, wherein the plurality of backbone fibers and the plurality of inter-layer fibers are biological fibers.

5. (canceled)

6. The biological fiber membrane of claim 1, further comprising an active ingredient or a drug.

7. The biological fiber membrane of claim 6, wherein the active ingredient is selected from the group consisting of a humectant, a whitening ingredient, an anti-wrinkle ingredient, an exfoliating ingredient, a growth factor and an enzyme.

8. (canceled)

9. The biological fiber membrane of claim 2, wherein the amount of the biological fibers per unit area in the biological fiber membrane is from 0.005 g/cm² to 0.008 g/cm².

10. The biological fiber membrane of claim 1 having a thickness of at least 20 μm.

11. The biological fiber membrane of claim 10, wherein the thickness is from 20 μm to 30 μm.

12. The biological fiber membrane of claim 2, wherein the diameter of each of the biological fiber is from 20 nm to 100 nm.

13. A method for preparing a biological fiber membrane, comprising:
providing a flat container having a culture medium having mannitol and agar, wherein the culture medium has a carbon source, a peptone and a yeast extract at a weight ratio from 5:1:1 to 4:1:1; and
stationarily culturing bacteria of the genus Gluconacetobacter in the culture medium at 25 to 28° C. for 24 hours to 96 hours, wherein the pH value of the culture medium is from 0.5 to 6.

14. (canceled)
